(19) United States
(12) Patent Application Publication
(10) Pub. No.: US 2016/0030367 A1
(43) Pub. Date: Feb. 4, 2016

(54) Methods and Compositions for Preventing Metastasis and for Improving the Survival Time

(71) Applicants: INSERM (INSTITUT NATIONAL DE LA SANTÉ ET DE LA RECHERCHE MÉDICALE), Paris (FR); UNIVERSITE NICE SOPHIA ANTIPOLIS, Nice (FR)

(72) Inventors: Stephane Rocchi, Nice Cedex 3 (FR); Robert Ballotti, Nice Cedex 3 (FR); Michael Cerezo, Nice Cedex 3 (FR)

(21) Appl. No.: 14/775,208

(22) PCT Filed: Mar. 14, 2014
(86) PCT No.: PCT/EP2014/055154

§ 371 Date: Sep. 11, 2015

(30) Foreign Application Priority Data
Mar. 15, 2013 (EP) 13305304.1

(51) Int. Cl. A61K 31/155; C12Q 1/68; G01N 33/68; A61K 45/06

(52) U.S. Cl. CPC A61K 31/155; A61K 45/06; C12Q 1/6886; G01N 33/6893; C12Q 2600/156; C12Q 2600/106; G01N 2800/7028; G01N 2800/52

(57) ABSTRACT

The invention relates to an AMPK activator (such as for instance metformin) for use in preventing metastasis in a patient suffering from a cancer, wherein said patient has a non-mutated p53 gene or lacks a mutant form of the p53 protein. The invention also relates to an AMPK activator for use in improving the survival time of a patient suffering from a cancer, wherein said patient has a non-mutated p53 gene or lacks a mutant form of the p53 protein.

### FIELD OF THE INVENTION

The invention relates to the field of oncology and cancer therapy. More particularly, the invention relates to an AMPK activator (such as metformin) for use in preventing metastasis in a patient suffering from a cancer, wherein said patient has a non-mutated p53 gene or lacks a mutant form of the p53 protein.

### BACKGROUND OF THE INVENTION

Many studies have been investigated for identifying efficient drugs useful for preventing metastasis and improving survival time of a patient suffering from a cancer such as melanoma since for instance, metastatic melanoma is one of the most aggressive and highly proliferative human malignancies with a median survival of only 6-9 months once distant sites become seeded from skin (1). Typically, primary lesions progress to malignant tumors through a multistep process including dysplasia, radial growth phase (RGP), invasive vertical growth phase (VGP), and metastasis. For invade across the basal lamina and spread into the body, melanoma cells will reactivate a program called epithelial mesenchimal transition (EMT) to enable them with mobility properties. In addition, to detach from the basal membrane, melanoma cells modify particularly the expression of cadherins and integrins. During malignant transformation, there is loss of expression of E-cadherin in favor of the N-cadherin (2, 3). These changes allow the melanocytes to escape the control of keratinocytes, and after crossing the stratum basale, to interact with new cell types such as fibroblasts or vascular endothelial cells which promote tumor progression and metastasis. Melanocytes cross the basal lamina thanks to the secretion of matrix MMPs (Metalloproteinases) such as collagenases MMP-2 and MMP-9, which will degrade collagen IV, a major constituent of the basal lamina and allow the melanoma cells to invade locally underlying dermis. Several transcription factors that belong to the Snail superfamily of zing-finger transcription factor including Snail/SNAI1 and Slug/SNAI2 are involved in this mechanism. For example, these two proteins are central regulators of EMT during neural crest cell migration and cancer (4, 5). In addition, it was shown in melanoma that Slug functions as a melanocyte-specific factor required for the strong metastatic propensity of this tumor (6). More interesting, Slug is a p53 target that antagonizes p53-mediated apoptosis (7) and invasion (8). Elevated mortality that is caused by melanoma is attributed to its strong propensity to form distal metastases in organs, such as lung, liver, brain, and bones, and its notorious resistance to all current therapeutics (9). The new important challenge was thus to discover new therapeutic drugs that inhibit melanoma cell proliferation but also exhibit anti-metastasis properties.

The oral antidiabetic drug, metformin belongs to the family of biguanide and is the most widely used antidiabetic drug in the world. This drug has been shown to inhibit the energy-sensitive AMPK-mTOR signaling pathway that leads to reduced protein synthesis and cell proliferation. Recent studies indicate that metformin can potentially be used as an efficient anticancer drug in various neoplasms such as prostate carcinoma, breast, lung and pancreas cancers (10, 11). These results were confirmed by retrospective epidemiological studies that reported a decrease in cancer risk in diabetic patients treated with metformin (12). In addition, metformin was reported by several groups, including ours, to inhibit the proliferation of melanoma cells (13-16). In a previous study, the inventors demonstrated that metformin dramatically impairs the growth of melanoma tumor in vitro and in vivo by inducing cell death by autophagy leading to massive apoptosis (13).

Identifying drugs useful for preventing metastases and for improving survival time of a patient suffering from a cancer such as metastatic melanoma, are highly needed. However, until now, the anti-invasive and anti-metastatic properties of metformin, independently of its effect on melanoma cell survival, have never been analyzed.

### SUMMARY OF THE INVENTION

In a first aspect, the invention relates to an in vitro method for predicting the responsiveness of a patient suffering from a cancer to a prophylactic treatment with an AMPK activator for use in preventing metastasis, said method comprising a step of determining the presence of a mutated p53 gene or a mutant form of the p53 protein in a biological sample obtained from said patient.

In a second aspect, the invention also relates to an AMPK activator for use in preventing metastasis in a patient suffering from a cancer, wherein said patient has a non-mutated p53 gene or lacks a mutant form of the p53 protein.

In a third aspect, the invention further relates to an AMPK activator for use in improving the survival time of a patient suffering from a cancer, wherein said patient has a non-mutated p53 gene or lacks a mutant form of the p53 protein.

In another aspect, the invention relates to a kit-of-part composition comprising an AMPK activator and a p53 recombinant protein or a polynucleotide encoding thereof.

In still another aspect, the invention relates to a kit-of-part composition comprising an AMPK activator and a p53 recombinant protein or a polynucleotide encoding thereof for simultaneous, separate or sequential use in preventing metastasis in a patient suffering from a cancer, wherein said patient has mutated p53 gene or a mutant form of the p53 protein.

### DETAILED DESCRIPTION OF THE INVENTION

The inventors have demonstrated the effect of metformin on melanoma invasion and metastasis development. Using different in vitro approaches, they have shown that metformin and inhibit cell invasion without affecting cell migration and independently of anti-proliferation action. This inhibition is correlated with modulation of expression of proteins involved in epithelial mesenchimal transition such as Slug, Snail, SPARC, fibronectin and N-Cadherin and with inhibition of MMP-2 and MMP-9 activation. Further they have underlined that this process is dependent of activation of AMPK and tumor suppressor protein, p53. Finally, they have shown that metformin inhibits melanoma metastasis development in mice using extravasation and metastasis models.

The inventors have also demonstrated the effect of others AMPK activators on melanoma invasion as well as the effect of AMPK activators including metformin on prostate and lung cancer invasion.

Once again, they have underlined that this process is dependent of activation of AMPK and tumor suppressor protein, p53 on p53-mutated cancer cell lines or not.

### DEFINITIONS

Throughout the specification, several terms are employed and are defined in the following paragraphs.

As used herein, the term “p53” refers to both p53 protein and the TP53 gene. The term “TP53” refers to the gene encoding p53 protein and the term “p53 protein” refers to a tumor suppressor protein that in humans is encoded by the TP53 gene. p53 is crucial in multicellular organisms, where it regulates multiple cellular process such as cell cycle arrest, cell death, senescence, metabolic pathways and other outcomes thereby acting as a tumor suppressor that is involved in preventing cancer. p53 is also known Cellular tumor antigen p53, Antigen NY-CO-13, Phosphoprotein p53, Transformation-related protein 53 (TRP53), Tumor suppressor p53. The transcription factor p53 is a 393-amino acids protein composed of 5 domains: a N-terminal transactivation domain (TAD), a proline-rich domain (PRD), a core DNA binding domain (DBD), a tetramerization domain (4D) and a C-terminal regulatory domain (CTD). The naturally occurring human p53 gene has a nucleotide sequence as shown in Genbank Accession number NM_{—}000546 and the naturally occurring human p53 protein has an aminoacid sequence as shown in Genbank Accession number NP_{—}000537.

The term “gene” includes the segment of DNA involved in producing a polypeptide chain. Specifically, a gene includes, without limitation, regions preceding and following the coding region, such as the promoter and 3′-untranslated region, respectively, as well as intervening sequences (introns) between individual coding segments (exons).

The terms “polypeptide” and “protein” are used interchangeably as a generic term referring to native protein, fragments, or variants of a polypeptide sequence. The term “polypeptide” does not exclude post-translational modifications that include but are not limited to phosphorylation, acetylation, glycosylation and the like.

The term “nucleic acid” or “polynucleotide” includes deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, and mRNA encoded by a gene.

“p53 mutations” refers to mutations in the p53 protein and p53 gene. Examples of TP53 mutations are well known from the art and are described for instance in, e.g., Soussi T. (2007) Cancer Cell 12(4):303-12; Cheung K. J. (2009) Br J Haematol. 146(3):257-69; Pfeifer G. P. et al. (2009) Hum Genet. 125(5-6):493-506; Petitjean A. et al. (2007) Oncogene 26(15):2157-65; Olivier et al., (2010) Cold Spring Harb Perspect Biol 2, a001008).

More particularly, SNPs in the TP53 coding sequence, leading to missense mutations, nonsense mutations or frameshifts, are the principal mode of p53 alteration in human cancers (Olivier et al., (2010) Cold Spring Harb Perspect Biol 2, a001008). It should be further noted that the functional importance of the p53 DNA-binding domain (DBD) is demonstrated by the fact that more than 70% of TP53 mutations are missense mutations affecting residues within this domain, and leading to a decreased capacity in target gene transactivation.

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein) expressed by a modified gene or DNA sequence. Generally a mutation is identified in a patient by comparing the sequence of a nucleic acid or polypeptide expressed by said patient with the corresponding nucleic acid or polypeptide expressed in a control population. A mutation in the genetic material may also be “silent”, i.e. the mutation does not result in an alteration of the amino acid sequence of the expression product. A “single nucleotide polymorphism” or “SNP” occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A SNP usually arises due to substitution of one nucleotide for another at the polymorphic site, and occurs in at least 1% of the population.

As used herein, the term “biological sample” includes any biological specimen obtained from a patient. Samples include, without limitation, whole blood, plasma, serum, red blood cells, white blood cells (e.g., peripheral blood mononuclear cells), saliva, urine, stool (i.e., feces), lymph, fine needle aspirate, any other bodily fluid, a tissue sample (e.g., tumor tissue) such as a biopsy of a tumor, and cellular extracts thereof. In some embodiments, the biological sample is a formalin fixed paraffin embedded (FFPE) tumor tissue sample, e.g., from a solid tumor. In certain embodiments, the biological sample is obtained by isolating circulating cells of a solid tumor from a whole blood cell pellet using any technique known in the art. As used herein, the term “circulating cancer cells” comprises cells that have either metastasized or micro metastasized from a solid tumor and includes circulating tumor cells, and cancer stem cells. In other embodiments, the biological sample is whole blood or a fractional component thereof such as plasma, serum, or a cell pellet.

A “nucleic acid sample” can be obtained from a patient using routine methods. Such samples comprise any biological matter from which nucleic acid can be prepared. As non-limiting examples, suitable samples include whole blood, serum, plasma, saliva, cheek swab, urine, or other bodily fluid or tissue that contains nucleic acid. In one embodiment, the methods of the present invention are performed using whole blood or fractions thereof such as serum or plasma, which can be obtained readily by non-invasive means and used to prepare genomic DNA. In another embodiment, genotyping involves the amplification of a patient's nucleic acid using PCR. Use of PCR for the amplification of nucleic acids is well known in the art (see, e.g., Mullis et al., The Polymerase Chain Reaction, Birkhauser, Boston, (1994). Generally, protocols for the use of PCR in identifying mutations and polymorphisms in a gene of interest are described in Theophilus et al., “PCR Mutation Detection Protocols,” Humana Press (2002). Further protocols are provided in Innis et al., “PCR Applications: Protocols for Functional Genomics,” 1st Edition, Academic Press (1999). Applicable PCR amplification techniques are described in Ausubel et al., Current Protocols in Molecular Biology, John Wiley & Sons, Inc., New York (1999); Theophilus et al., “PCR Mutation Detection Protocols,” Humana Press (2002); and Innis et al., “PCR Applications: Protocols for Functional Genomics,” 1st Edition, Academic Press (1999). General nucleic acid hybridization methods are described in Anderson, “Nucleic Acid Hybridization,” BIOS Scientific Publishers (1999). Amplification or hybridization of a plurality of transcribed nucleic acid sequences (e.g., mRNA or cDNA) can also be performed using mRNA or cDNA sequences arranged in a microarray. Microarray methods are generally described in Hardiman, “Microarrays Methods and Applications: Nuts & Bolts,” DNA Press (2003) and Baldi et al., “DNA Microarrays and Gene Expression: From Experiments to Data Analysis and Modeling,” Cambridge University Press (2002).

As used herein, the term “prophylactic treatment” refers to preventative measures, wherein the object is to prevent or slow down (lessen) an undesired physiological change or disorder, such as the spread of cancer (e.g., invasion and metastasis development). For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having cancer and those having high risk of developing metastasis (e.g., patients suffering from melanoma).

An “effective amount” of a drug is an amount that produces the desired effect.

A “prophylactically effective amount” of a drug is an amount of a drug that, when administered to a patient, will have the intended prophylactic effect, e.g., preventing or delaying the onset of the disease or symptoms, or reducing the likelihood of the onset of the disease or symptoms. The full prophylactic effect does not necessarily occur by administration of one dose and may occur only after administration of a series of doses. Thus, a prophylactically effective amount may be administered in one or more administrations.

The term “cancer” is intended to include any member of a class of diseases characterized by the uncontrolled growth of aberrant cells. The term includes all known cancers and neoplastic conditions, whether characterized as malignant, benign, soft tissue, or solid, and cancers of all stages and grades including pre- and post-metastatic cancers. Examples of cancer include, but are not limited to, carcinoma, blastoma, sarcoma, lymphoma and leukemia. More particular examples of such cancers include, but are not limited to, skin cancer (melanoma); lung cancer (e.g., non-small cell lung cancer); digestive and gastrointestinal cancers such as colorectal cancer, small intestine cancer, and stomach (gastric) cancer; esophageal cancer; bladder cancer; liver cancer; pancreatic cancer; breast cancer; ovarian cancer; prostate cancer: renal cancer (e.g., renal cell carcinoma); cancer of the central nervous system; head and neck cancers; osteogenic sarcomas. As used herein, a “tumor” comprises one or more cancer cells or benign cells or precancerous cells.

By “metastasis” or “tumor metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream or lymphatics, and stopping at a distant site. At the new site, the cells establish a blood supply and can grow to form a life-threatening mass. In certain embodiments, the term metastatic tumor refers to a tumor that is capable of metastasizing, but has not yet metastasized to tissues or organs elsewhere in the body. In certain embodiments, the term metastatic tumor refers to a tumor that has metastasized to tissues or organs elsewhere in the body.

By “primary tumor” or “primary cancer” is meant the original cancer and not a metastatic lesion located in another tissue, organ, or location in the patient's body.

The term “AMPK” refers to the 5′ adenosine monophosphate-activated protein kinase which is an important regulatory protein for cellular energy balance and which is considered a master switch of glucose and lipid metabolism in various organs, especially in skeletal muscle and liver. The heterotrimeric protein AMPK is formed by α, β, and γ subunits.

The term “AMPK activator” refers to any compound (natural or synthetic) which increases AMPK activity (e.g. by promoting phosphorylation at Thr-172 on the a subunit). AMPK activity may be measured by an assay as described in Gorton, et al., Eur. J. Biochem. 1995, 229:558-565). AMPK activators are well known in the art (see for review Zhang et al, Cell Metabolism 9, May 6, 2009 or Gruzman et al, Review of Diabetic Studies (2009) 6:13-36). Activation of AMPK may be induced by indirect activators such as biguanide derivatives (metformin) or thiazolidinediones (troglitazone, rosiglitazone or pioglitazone). Alternatively, activation of AMPK may be induced by direct activators such as A-769662 (Cool, B., et al. (2006). Cell Metab. 3, 403-416) or PT1 (Pang et al. (2008) J. Biol. Chem. 283, 16051-16060).

The term “patient” refers to a human being. Typically, the patient suffering from a cancer to be tested in the context of the invention is a patient having malignant tumors (for instance malignant solid tumors such as melanoma) and may be under chemotherapy treatment. Said “cancer patients” may be ambulatory patients (outpatients) or hospitalized patients. Preferably, patients are at risk for developing metastasis and have not being initially diagnosed with metastatic cancer. Patients may also suffer from a recurrent cancer.

### Predictive Methods of the Invention

In a first aspect, the invention relates to an in vitro method for predicting the responsiveness of a patient suffering from a cancer to a prophylactic treatment with an AMPK activator for use in preventing metastasis, said method comprising a step of determining the presence of a mutated p53 gene or a mutant form of the p53 protein in a biological sample obtained from said patient.

In one embodiment, the presence of a mutated p53 gene or a mutant form of the p53 protein in said biological sample is indicative of the non-response of the patient to the prophylactic treatment with an AMPK activator.

In one embodiment, the biological sample obtained from the patient is a tumor biopsy.

In still another embodiment, the mutation is detected by using an amplification assay, a hybridation assay, by molecular cloning and sequencing, by microarray analysis or by any method used for determining the presence of a mutation within a DNA sequence or of a mutated form of a protein.

In a particular embodiment, the p53 gene present in the biological sample is amplified by polymerase chain reaction (PCR) or by ligase chain reaction (LCR).

In another particular embodiment, a DNA hybridization assay is used to detect the p53 gene in the biological sample.

Identifying Patients with p53 Mutations:

Alterations of a wild-type p53 gene according to the present invention encompass all forms of mutations such as insertions, inversions, deletions, and/or point mutations. Somatic mutations are those which occur only in certain tissues, e.g., in the tumor tissue, and are not inherited in the germ line. If only a single allele is somatically mutated, an early neoplastic state is indicated. However, if both alleles are mutated then a late neoplastic state is indicated. Germ line mutations can be found in any of a body's tissues. The finding of p53 mutations in a benign tumor is also a condition that can be treated prophylactically.

As previously mentioned, examples of TP53 mutations are described in, e.g., Soussi T. (2007) Cancer Cell 12(4):303-12; Cheung K. J. (2009) Br J Haematol. 146(3):257-69; Pfeifer G. P. et al. (2009) Hum Genet. 125(5-6):493-506; Petitjean A. et al. (2007) Oncogene 26(15):2157-65; Olivier et al., (2010) Cold Spring Harb Perspect Biol 2, a001008.

As previously mentioned, 70% of TP53 mutations are missense mutations affecting residues within the p53 DNA-binding domain (DBD).

Patients suffering from a cancer (and precancerous lesions) that can be prophylactically treated with an AMPK activator include patient suffering from any cancer whether said patient has a non-mutated p53 gene or lacks a mutant form of the p53 protein.

Such cancers include melanoma, breast cancer, neuroblastoma, gastrointestinal carcinoma such as rectum carcinoma, colon carcinoma, familial adenomatous polyposis carcinoma and hereditary non-polyposis colorectal cancer, esophageal carcinoma, labial carcinoma, larygial carcinoma, hypopharyngial carcinoma, tongue carcinoma, salivary gland carcinoma, gastric carcinoma, medullary thyroid carcinoma, papillary thyroid carcinoma, renal carcinoma, kidney parenchymal carcinoma, ovarian carcinoma, cervical carcinoma, uterine corpus carcinoma, endometrium carcinoma, choriocarcinoma, pancreatic carcinoma, prostate carcinoma, testis carcinoma, urinary carcinoma, brain tumors such as glioblastoma, astrocytoma, meningioma, medulloblastoma and peripheral neuroectodermal tumors, Hodgkin's lymphoma, non-Hodgkin's lymphoma, Burkitt's lymphoma, acute lymphocytic leukemia (ALL), chronic lymphocytic leukemia (CLL), acute myelologenous leukemia (AML), chronic myelologenous leukemia (CML), adult T-cell leukemia/lymphoma, hepatocellular carcinoma, gallbladder carcinoma, bronchial carcinoma, small cell lung carcinoma, non-small cell lung carcinoma, multiple myeloma, basal cell carcinoma, teratoma, retinoblastoma, choroidal melanoma, seminoma, rhabdomyosarcoma, craniopharyngioma, osteosarcoma, chondrosarcoma, myosarcoma, liposarcoma, fibrosarcoma, Ewing's sarcoma and plasmocytoma. Particular tumors include those of the skin, brain, liver, kidney, bladder, breast, gastric, ovarian, colorectal, prostate, pancreatic, lung, vulval, thyroid, colorectal, oesophageal, sarcomas, glioblastomas, head and neck, leukemias and lymphoid malignancies.

Mutant p53 genes or gene products (i.e. mutant forms of the p53 protein) can be detected in tumor samples or, in some types of cancer, in biological samples such as urine, stool, sputum or serum. For example, TP53 mutations can often be detected in urine for bladder cancer and prostate cancer, sputum for lung cancer, or stool for colorectal cancer. Cancer cells are found in blood and serum for cancers such as lymphoma or leukemia. The same techniques discussed above for detection of mutant p53 genes or gene products (i.e. mutant forms of the p53 protein) in tumor samples can be applied to other body samples.

A p53 (TP53) gene mutation in a biological sample can be identified using any method known in the art. One of the most commonly used methods to “identify” p53 mutants is by utilizing immunohistochemistry (IHC) on tumor sections stained with a p53 antibody. Positive staining with an antibody against p53 is often used as a surrogate for sequencing the gene itself. Some have proposed combining sequencing and IHC, since p53 mutants that are highly expressed tend to be more oncogenic.

In one embodiment, nucleic acid from the biological sample is contacted with a nucleic acid probe that is capable of specifically hybridizing to nucleic acid encoding a mutated p53 protein, or fragment thereof incorporating a mutation, and detecting the hybridization. In a particular embodiment the probe is detectably labelled such as with a radioisotope, a fluorescent agent (rhodamine, fluoresceine) or a chromogenic agent. In a particular embodiment the probe is an antisense oligomer. The probe may be from about 8 nucleotides to about 100 nucleotides, or about 10 to about 75, or about 15 to about 50, or about 20 to about 30. Kits for identifying p53 mutations in a biological sample are available that include an oligonucleotide that specifically hybridizes to or adjacent to a site of mutation in the p53 gene. The p53 Amplichip™ developed by Roche is a good example of this technology; http://www.ncbi.nlm.nih.gov/pmc/articles/PMC2691672/?tool=pubmed.

A mutation in the p53 gene in a biological sample can be detected by amplifying nucleic acid corresponding to the p53 gene obtained from the biological sample, or a biologically active fragment, and comparing the electrophoretic mobility of the amplified nucleic acid to the electrophoretic mobility of corresponding wild-type p53 gene or fragment thereof. A difference in the mobility indicates the presence of a mutation in the amplified nucleic acid sequence. Electrophoretic mobility may be determined on polyacrylamide gel. Alternatively, an amplified p53 gene or fragment nucleic acid may be analyzed for detection of mutations using Enzymatic Mutation Detection (EMD) (Del Tito et al, Clinical Chemistry 44:731-739, 1998). EMD uses the bacteriophage resolvase T4 endonuclease VII, which scans along double-stranded DNA until it detects and cleaves structural distortions caused by base pair mismatches resulting from point mutations, insertions and deletions. Detection of two short fragments formed by resolvase cleavage, for example by gel eletrophoresis, indicates the presence of a mutation. Benefits of the EMD method are a single protocol to identify point mutations, deletions, and insertions assayed directly from PCR reactions eliminating the need for sample purification, shortening the hybridization time, and increasing the signal-to-noise ratio. Mixed samples containing up to a 20-fold excess of normal DNA and fragments up to 4 kb in size can been assayed. However, EMD scanning does not identify particular base changes that occur in mutation positive samples requiring additional sequencing procedures to identity of the mutation if necessary. CEL I enzyme can be used similarly to resolvase T4 endonuclease VII as demonstrated in U.S. Pat. No. 5,869,245.

In order to detect the mutation of the wild-type p53 gene, a biological sample such as a biopsy of the tumor or a sample comprising cancer cells or precancerous cells (such as blood, serum, CSF, stool, urine or sputum) is obtained by methods well known in the art and appropriate for the particular type and location of the tumor. For instance, samples of skin cancer lesions may be obtained by resection, or fine needle aspiration. Means for enriching a tissue preparation for tumor cells are known in the art. For example, the tissue may be isolated from paraffin or cryostat sections. Cancer cells may also be separated from normal cells by flow cytometry or laser capture microdissection. These as well as other techniques for separating tumor from normal cells are well known in the art. If the tumor tissue is highly contaminated with normal cells, detection of mutations is more difficult.

Detection of point mutations may be accomplished by molecular cloning of the p53 allele (or alleles) and sequencing that allele(s) using techniques well known in the art. Alternatively, the polymerase chain reaction (PCR) can be used to amplify gene sequences directly from a genomic DNA preparation from the tumor tissue. The DNA sequence of the amplified sequences can then be determined and mutations identified. The polymerase chain reaction is the preferred method and it is well known in the art and described in Saiki et al., Science 239:487, 1988; U.S. Pat. Nos. 4,683,203; and 4,683, 195.

Primer sequences and amplification protocols for evaluating p53 mutations are known to those in the art and have been published. For a list of primer sequences used to sequence p53, refer to: Reles et al. Correlation of p53 Mutations with Resistance to Platinum-based Chemotherapy and Shortened Survival in Ovarian Cancer. Clinical Cancer Research (2001).

The ligase chain reaction (LCR), which is known in the art, can also be used to amplify p53 sequences. See Wu et al., Genomics, Vol. 4, pp. 560-569 (1989). In addition, a technique known as allele specific PCR can be used. (See Ruano and Kidd, Nucleic Acids Research, Vol. 17, p. 8392, 1989.) According to this technique, primers are used which hybridize at their 3′ends to a particular p53 mutation. If the particular p53 mutation is not present, an amplification product is not observed. Amplification Refractory Mutation System (ARMS) can also be used as disclosed in European Patent Application Publication No. 0332435 and in Newton et al., Nucleic Acids Research, Vol. 17, p. 7, 1989. Insertions and deletions of genes can also be detected by cloning, sequencing and amplification. In addition, restriction fragment length polymorphism, (RFLP) probes for the gene or surrounding marker genes can be used to score alteration of an allele or an insertion in a polymorphic fragment. Single stranded conformation polymorphism (SSCP) analysis can also be used to detect base change variants of an allele. (Orita et al., Proc. Natl. Acad. Sci. USA Vol. 86, pp. 2766-2770, 1989, and Genomics, Vol. 5, pp. 874-879, 1989.) Other techniques for detecting insertions and deletions as are known in the art can be used.

Mismatches, according to the invention are hybridized nucleic acid duplexes which are not 100% complementary. The lack of total complementarity may be due to deletions, insertions, inversions, substitutions or frameshift mutations. Mismatch detection can be used to detect point mutations in the gene or its mRNA product. While these techniques are less sensitive than sequencing, they are simpler to perform on a large number of tumor samples. An example of a mismatch cleavage technique is the RNase protection method, which is described in detail in Winter et al., Proc. Natl. Acad. Sci. USA, Vol. 82, p. 7575, 1985 and Meyers et al., Science, Vol. 230, p. 1242, 1985. A labelled riboprobe which is complementary to the human wild-type p53 gene coding sequence can also be used. The riboprobe and either mRNA or DNA isolated from the tumor tissue are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mRNA or DNA. The riboprobe need not be the full length of the p53 mRNA or gene. If the riboprobe comprises only a segment of the p53 mRNA or gene it will be desirable to use a number of these probes to screen the whole mRNA sequence for mismatches.

In a similar manner, DNA probes can be used to detect mismatches, through enzymatic or chemical cleavage. See, e.g., Cotton et al., Proc. Natl. Acad. Sci. USA, Vol. 85, 4397, 1988; and Shenk et al., Proc. Natl. Acad. Sci. USA, Vol. 72, p. 989, 1975. Alternatively, mismatches can be detected by shifts in the electrophoretic mobility of mismatched duplexes relative to matched duplexes. See, e.g., Cariello, Human Genetics, Vol. 42, p. 726, 1988. With either riboprobes or DNA probes, the cellular mRNA or DNA which might contain a mutation can be amplified using PCR before hybridization. Changes in DNA of the p53 gene can also be detected using Southern hybridization, especially if the changes are gross rearrangements, such as deletions and insertions.

DNA sequences of the p53 gene which have been amplified by use of polymerase chain reaction may also be screened using allele-specific probes. These probes include nucleic acid oligomers, each of which contains a region of the p53 gene sequence harboring a known mutation. For example, one oligomer may be about 30 nucleotides in length, corresponding to a portion of the p53 gene sequence. By use of a battery of such allele-specific probes, PCR amplification products can be screened to identify the presence of a previously identified mutation in the p53 gene. Hybridization of allele-specific probes with amplified p53 sequences can be performed, for example, on a nylon filter. Hybridization to a particular probe under stringent hybridization conditions indicates the presence of the same mutation in the tumor tissue as in the allele-specific probe. This is used with the p53 Amplichip described above.

Alteration of wild-type p53 genes can also be detected by screening for alteration of wild-type p53 protein. For example, monoclonal antibodies immunoreactive with p53 can be used to screen a tissue. As mentioned above, one of the common ways to “detect” p53 mutations is to see strong p53 immunostaining in tissue sections (these are not mutant p53 specific antibodies, but simply take advantage of the fact that most mutant p53 proteins are more stable (and thus more abundant) than wild-type p53. Antibodies specific for products of mutant alleles could also be used to detect mutant p53 gene product. Such immunological assays can be done in any convenient format known in the art. These include Western blots, immunohistochemical assays and ELISA assays. Any means for detecting an altered p53 protein or p53 mRNA can be used to detect alteration of wild-type p53 genes or the expression product of the gene. Point mutations may be detected by amplifying and sequencing the mRNA or via molecular cloning of cDNA made from the mRNA (or by sequencing genomic DNA). The sequence of the cloned cDNA can be determined using DNA sequencing techniques which are well known in the art. The cDNA can also be sequenced via the polymerase chain reaction (PCR).

### Therapeutic Methods and Uses

The invention provides methods and compositions (such as pharmaceutical compositions) for preventing metastasis. The invention also provides methods and compositions for inhibiting or preventing invasion of cancer cells. The invention further provides methods and compositions for improving the survival time of a patient.

Accordingly, in a second aspect, the invention relates to an AMPK activator for use in preventing metastasis in a patient suffering from a cancer, wherein said patient has a non-mutated p53 gene or lacks a mutant form of the p53 protein.

In one embodiment, the AMPK activator is selected from the group consisting of biguanide derivatives, stilbene derivatives, thiazolidinedione (TZD) derivatives, 5-aminoimidazole-4-carboxamide-1-β-D-ribofuranoside (AICAR), thienopyridone derivatives, imidazole derivatives and thiazole derivatives.

In one particular embodiment, the biguanide derivative is a compound of formula (I) as disclosed in the patent application WO2011147528:

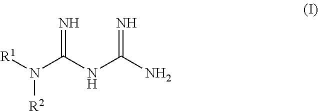

in which
R¹, R² each, independently of one another, denote H, A, Alk, (CH2)nAr, (CH2)nCyc or (CH2)nHet,
R¹ and R² together also denote an alkylene chain having 2, 3, 4, 5 or 6 C atoms, in which one CH₂ group may be replaced by O, S, SO, SO₂, NH, NR⁸, NCOR⁸ or NCOOR⁸,
   and wherein the alkylene chain is unsubstituted or mono-, di- or trisubstituted by Hal, A, OR¹¹, N(R¹¹)₂, NO₂, CN, phenyl, ═O, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
A denotes unbranched or branched alkyl having 1-10 C atoms, in which one, two or three CH and/or CH₂ groups may be replaced by O, S, SO, SO₂, NH, NR⁸ and/or by —CH═CH— groups and/or, in addition, 1-5 H atoms may be replaced by F, Cl, Br and/or R⁷,
Cyc cycloalkyl having 3-7 C atoms,
Alk denotes alkenyl or alkinyl having 2-6 C atoms,
R⁷ denotes COOR⁹, CONR⁹R¹⁰, NR⁹R¹⁰, NHCOR⁹, NHCOOR⁹ or OR⁹,
R⁸ denotes cycloalkyl having 3-7 C atoms,
   cycloalkylalkylene having 4-10 C atoms,
   Alk or
   unbranched or branched alkyl having 1-6 C atoms,
R⁹, R¹⁰ each, independently of one another, denote H or alkyl having 1-6 C atoms, in which 1-3 CH₂ groups may be replaced by O, S, SO, SO₂, NH, NMe or NEt and/or, in addition, 1-5H atoms may be replaced by F and/or Cl,
Ar denotes phenyl, naphthyl or biphenyl, each of which is un-substituted or mono-, di- or trisubstituted by Hal, A, OR¹¹, N(R¹¹)₂, NO₂, CN, phenyl, CON(R¹¹)₂, NR¹¹COA, NR¹¹CON(R¹¹)₂, NR¹¹SO₂A, COR¹¹, SO₂N(R¹¹)₂, S(O)ₘA, —[C(R¹¹)₂]ₙ—COOR¹¹ and/or —O[C(R¹¹)₂]ₒ—COOR¹¹,
Het denotes a mono- or bicyclic saturated, unsaturated or aromatic heterocycle having 1 to 4 N, O and/or S atoms, which may be mono-, di- or trisubstituted by Hal, A, OR¹¹, N(R¹¹)₂, NO₂, CN, COOR¹¹, CON(R¹¹)₂, NR¹¹COA, NR¹¹SO₂A, COR¹¹, SO₂NR¹¹, S(O)ₘA, ═S, ═NR¹¹ and/or ═O (carbonyl oxygen),
R¹¹ denotes H or A,
Hal denotes F, Cl, Br or I,
m denotes 0, 1 or 2,
n denotes 0, 1, 2, 3 or 4,
o denotes 1, 2 or 3,
with the exclusion of the compounds of formula I in which:
   a—R¹═H, R²═H;
   b—R¹═H, R²=phenethyl;
and pharmaceutically usable salts, solvates, tautomers and stereoisomers thereof, including mixtures thereof in all ratios.

In a preferred embodiment, the biguanide derivative is metformin or phenformin.

In a still preferred embodiment, metformin is administered to the patient suffering from a cancer at a dose equal to those administered to diabetic patients (3 g/75 kg/day).

In one particular embodiment, the AMPK activator is a stilbene derivative (e.g. a hydroxystilbene). An example of patent application disclosing stilbene derivatives (such as trihydroxystilbenes) is EP0953344.

In a preferred embodiment, the stilbene derivative is resveratrol (3,5,4′-trihydroxy-trans-stilbene).

In one particular embodiment, the AMPK activator is a TZD derivative. An example of patent application disclosing TZD derivative is U.S. Pat. No. 4,687,777.

In a preferred embodiment, the TZD derivative is troglitazone, rosiglitazone or pioglitazone.

Other examples of patent applications disclosing AMPK activators are WO2009135580, WO2009124636, US20080221088, or EP1754483 which all disclose thienopyridone derivatives, WO2008120797, EP2040702 which discloses imidazole derivatives, EP1907369 which discloses thiazole derivatives.

In one embodiment, the patient suffering from cancer is treated with a chemotherapeutic agent against said cancer.

As used herein, the term “chemotherapeutic agent” refers to any compound (natural or synthetic), primarily a cytotoxic or cytostatic agent, that is used to treat a condition, particularly cancer. As used herein, the term “cytostatic agents” are mechanism-based agents that slow the progression of neoplastic disease and include drugs, biological agents, and radiation. As used herein the term “cytotoxic agents” are any agents or processes that kill neoplastic cells and include drugs, biological agents, and radiation. In addition, the term “cytotoxic” is inclusive of the term “cytostatic”.

Chemotherapeutic agents include, for example, fluropyrimidines; pyrimidine nucleosides; purine nucleosides; anti-folates, platinum agents; anthracyclines/anthracenediones; epipodophyllotoxins; camptothecins; hormones; hormonal complexes; antihormonals; enzymes, proteins, peptides and polyclonal and/or monoclonal antibodies; vinca alkaloids; taxanes; epothilones; antimicrotubule agents; alkylating agents; antimetabolites; topoisomerase inhibitors; and various other cytotoxic and cytostatic agents.

In one embodiment, the patient is treated with a chemotherapeutic agent against melanoma (such as dacarbazine such as (DTIC), temozolomide (Temodar), fotemustine (Muphoran), vindesine (Eldisine), ipilimumab (Yervoy) and vemurafenib (Zelboraf)).

Another aspect of the present invention relates to a method for preventing metastasis in a patient suffering from a cancer, wherein said patient has a non-mutated p53 gene or lacks a mutant form of the p53 protein, comprising administering to said patient a prophylactically effective amount of an AMPK activator.

The invention also relates to a method for preventing metastasis in a patient suffering from a cancer, comprising the steps of:
a) providing a biological sample from said patient,
b) determining the presence of a mutated p53 gene or a mutant form of the p53 protein in said biological sample,
c) administering to the patient a prophylactically effective amount of an AMPK activator, if no mutated p53 gene or no mutant form of the p53 protein is present in said biological sample.

In a third aspect, the invention relates to an AMPK activator for use in improving the survival time of a patient suffering from a cancer, wherein said patient has a non-mutated p53 gene or lacks a mutant form of the p53 protein.

As used herein, the term “survival” refers to the patient remaining alive, and includes overall survival (OS) as well as progression free survival (PFS).

As used herein, the term “overall survival” refers to the patient remaining alive for a defined period of time, such as 1 year, 5 years, etc from the time of diagnosis or treatment.

As used herein, the term “progression free survival” refers to the patient remaining alive, without the cancer progressing or getting worse.

As used herein, the term “improving the survival time” is meant increasing overall or progression free survival in a treated patient relative to an untreated patient (i.e. relative to a patient not treated with AMPK activator, such as metformin).

Suitable AMPK activators have been described above.

In one embodiment, the patient suffering from cancer is treated with a chemotherapeutic agent against said cancer as described above.

Another aspect of the present invention relates to a method for improving the survival time of a patient suffering from a cancer, wherein said patient has a non-mutated p53 gene or lacks a mutant form of the p53 protein, comprising administering to said patient a prophylactically effective amount of an AMPK activator.

The invention also relates to a method for improving the survival time in a patient suffering from a cancer, comprising the steps of:
a) providing a biological sample from said patient,
b) determining the presence of a mutated p53 gene or a mutant form of the p53 protein in said biological sample,
c) administering to the patient a prophylactically effective amount of an AMPK activator, if no mutated p53 gene or no mutant form of the p53 protein is present in said biological sample.

### Pharmaceutical Compositions

Another aspect of the invention is a pharmaceutical composition for use in preventing metastasis comprising an AMPK activator as described above and a pharmaceutically acceptable carrier.

Any AMPK activator of the invention as above described may be combined with pharmaceutically acceptable excipients, and optionally sustained-release matrices, such as biodegradable polymers, to form pharmaceutical compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc. The pharmaceutical compositions of the invention can be formulated for a topical, oral, intranasal, intraocular, intravenous, intramuscular or subcutaneous administration and the like.

The doses used for the administration can be adapted as a function of various parameters, and in particular as a function of the mode of administration used, of the relevant pathology, or alternatively of the desired duration of treatment. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

To prepare pharmaceutical compositions, an effective amount of an AMPK activator according to the invention may be dissolved or dispersed in a pharmaceutically acceptable carrier or aqueous medium. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the compositions can be brought about by the use in the compositions of agents delaying absorption (e.g. aluminium monostearate and gelatine). The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, mixtures thereof and in oils. Under ordinary conditions of storage and use, these preparations contain a preservative to prevent the growth of microorganisms.

The AMPK activators according to the invention can be formulated into a composition in a neutral or salt form. Pharmaceutically acceptable salts include the acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

### Kit-of-Part Compositions

Moreover, for patients having mutated p53 gene or a mutant form of the p53 protein in order to obtain a response to a prophylactic treatment with an AMPK activator to administer to said patient a p53 recombinant protein or a polynucleotide encoding thereof.

Thus, in another aspect, the invention relates to a kit-of-part composition comprising an AMPK activator and a p53 recombinant protein or a polynucleotide encoding thereof.

In one embodiment, said AMPK activator is one compound described above.

In one embodiment, said p53 recombinant protein or a polynucleotide encoding thereof is cell permeable p53 recombinant protein as described above in the patent application No US2012122796.

In another aspect, the invention also relates to present invention relates to an AMPK activator and a p53 recombinant protein or a polynucleotide encoding thereof for simultaneous, separate or sequential use in preventing metastasis in a patient suffering from a cancer, wherein said patient has mutated p53 gene or a mutant form of the p53 protein.

Accordingly, the invention also relates to a method for preventing metastasis in a patient suffering from a cancer, comprising the steps of:
a) providing a biological sample from said patient,
b) determining the presence of a mutated p53 gene or a mutant form of the p53 protein in said biological sample,
c) administering simultaneously, separately or sequentially to said patient a prophylactically effective amount of an AMPK activator and a p53 recombinant protein or a polynucleotide encoding thereof, if a mutated p53 gene or a mutant form of the p53 protein is present in said biological sample.

In still another aspect, the invention further relates to a kit-of-part composition comprising an AMPK activator and a p53 recombinant protein or a polynucleotide encoding thereof for simultaneous, separate or sequential use in improving the survival time of a patient suffering from a cancer, wherein said patient has mutated p53 gene or a mutant form of the p53 protein.

Accordingly, the invention further relates to a method for improving the survival time of a patient suffering from a cancer, comprising the steps of:
a) providing a biological sample from said patient,
b) determining the presence of a mutated p53 gene or a mutant form of the p53 protein in said biological sample,
c) administering simultaneously, separately or sequentially to said patient a prophylactically effective amount of an AMPK activator and a p53 recombinant protein or a polynucleotide encoding thereof, if a mutated p53 gene or a mutant form of the p53 protein is present in said biological sample.

The terms “kit”, “product” or “combined preparation”, as used herein, define especially a “kit-of-part” composition in the sense that the combination partners as defined above can be dosed independently or by use of different fixed combinations with distinguished amounts of the combination partners, i.e. simultaneously or at different time points. The parts of the kit-of-part can then, e.g., be administered simultaneously or chronologically staggered, that is at different time points and with equal or different time intervals for any part of the kit-of-part. The ratio of the total amounts of the combination partners to be administered in the combined preparation can be varied. The combination partners can be administered by the same route or by different routes. When the administration is sequential, the first partner may be for instance administered 1, 2, 3, 4, 5, 6, 12, 18 or 24 h before the second partner.

The present invention will be further illustrated by the following figures and examples. However, these examples and figures should not be interpreted in any way as limiting the scope of the present invention.

### FIGURES

FIG. 1: Effects of metformin on melanoma invasion. Invasion assay in coated Boyden chambers with 1205Lu, A375 and freshly isolated from patient melanoma cells treated 24 h with metformin (at the indicated concentrations) or PBS were performed. The results are expressed as percentages of the control. The bars indicate the mean±SD of triplicate samples. *, P<0.05; **, P<0.01; and ***, P<0.001.

FIG. 2: Effects of metformin on EMT markers: 1205Lu (A), A375 (B) and freshly isolated from patient melanoma cells (C) were treated 24 h with metformin (at the indicated concentrations) or PBS, lysed and analyzed by western-blot with Fibronectin, N-Cadherin, Vimentin, Sparc, Slug and Snail antibodies. HSP90 was used for load control.

FIG. 3: Effects of metformin on Matrix metalloproteinase. (A) Matrix metalloproteinase activity was measured on the culture media of 1205Lu melanoma cells treated 24 h with 10 mM of metformin or PBS. The results are expressed in arbitrary units. The bars indicate the mean±SD of triplicate samples. *, P<0.05; **, P<0.01; and ***, P<0.001. (B) ImageJ quantifications of three independent experiment of substrate zymography were shown. The results are expressed as percentages of the control. The bars indicate the mean±SD of triplicate samples. *, P<0.05; **, P<0.01; and ***, P<0.001.

FIG. 4: Implication AMPKα in the metformin effects. (A) 1205Lu melanoma cells were infected with adenovirus encoding a dominant negative form of α1 and α2 subunits of AMPK (Ad AMPK-DN) or an adenovirus control (Ad control). 24 h after infection, cells were treated with metformin (at the indicated concentrations) or PBS for 24 h, lysed and analysed by western-blot with Phospho-Acetyl-CoA Carboxylase (Ser79), Acetyl-CoA Carboxylase, α1 and α2 AMPK subunits and Slug antibodies. HSP90 was used for load control. (B) Invasion assay in coated Boyden chambers were performed on 1205Lu melanoma cells infected with adenovirus encoding a dominant negative forms of AMPKα (Ad AMPK-DN) or an adenovirus control (Ad control), treated or not 24 h with metformin. The results are expressed as percentages of the control. The bars indicate the mean±SD of triplicate samples. *, P<0.05; **, P<0.01; and ***, P<0.001.

FIG. 5: Implication of p53 in the anti-melanoma effects of metformin. (A) Invasion assay in coated Boyden chambers were performed on 1205Lu melanoma cells transfected with siRNA against p53 (sip53) or a siRNA control (siCtl) and treated 24 h with metformin (at the indicated concentrations) or PBS. The results are expressed as percentages of the control. The bars indicate the mean±SD of triplicate samples. *, P<0.05; **, P<0.01; and ***, P<0.001. (B) Invasion assay in coated Boyden chambers was performed on Mewo melanoma cells (mutated for p53) infected with adenovirus encoding a WT form of p53 (Adp53) or a control adenovirus (AdCtl) and treated with metformin (at the indicated concentrations) or PBS for 24 h. The results are expressed as percentages of the control. The bars indicate the mean±SD of triplicate samples. *, P<0.05; **, P<0.01; and ***, P<0.001.

FIG. 6: Effects of metformin on melanoma invasion in vivo. (A) 1205Lu melanoma cells were treated for 24 h with 10 mM of metformin or PBS and labeled with Green Cell Tracker and then injected via the tail vein in nude mice. After 24 h the lungs of the mice were imaged and the number of micro-metastasis was counted. (B and C) After injection of 1×10⁶ 1205Lu melanoma cells expressing luciferase into the tail vein, the nude mice were treated or not with metformin (60 mg/kg) for 39 days. The bioluminescence resulting from the presence of lung metastasis was quantified with a Photon Imager. The results after 7 days were quantified and presented in B. Quantification after 39 days were presented in C. The bars indicate the mean±SD of triplicate samples. *, P<0.05; **, P<0.01; and ***, P<0.001.

FIG. 7A: Effects of other AMPK activators on melanoma invasion. Invasion assay in coated Boyden chambers with 1205Lu and A375 treated 24 h with different AMPK activators (at the indicated concentrations) or PBS and DMSO were performed. The results are expressed as percentages of the control. The bars indicate the mean±SD of triplicate samples.

FIG. 7B: Effects of AMPK activators on prostate cancer invasion. Invasion assay in coated Boyden chambers with prostate cancer cells harboring (PC3) or not mutation (LNCap) in TP53 gene treated 24 h with different AMPK activators (at the indicated concentrations) or PBS were performed. The results are expressed as percentages of the control. The bars indicate the mean±SD of triplicate samples.

FIG. 8: Effects of AMPK activators on lung cancer invasion. Invasion assay in coated Boyden chambers with lung cancer cells harboring no mutation (A459) in TP53 gene treated 24 h with different AMPK activators (at the indicated concentrations) or PBS were performed. The results are expressed as percentages of the control. The bars indicate the mean±SD of triplicate samples.

### EXAMPLES

### Example 1

### Metformin Blocks Melanoma Invasion and Metastasis Development in a p53-Dependent Manner

Material & Methods

Reagents and Antibodies:

Metformin and other AMPK activators were purchased from Sigma-Aldrich (Saint Quentin Fallavier, France). Dulbecco's Modified Eagle's Medium (DMEM), penicillin/streptomycin and trypsin were from Invitrogen (Pontoise, France) and, fetal calf serum (FCS) from Hyclone (Brevieres, France). Slug, Snail, p53, HSP90, AMPKα1, AMPKα2 antibodies were purchased from Santa Cruz Biotechnology (TEBU; Le Perray en Yvelines, France). Anti-AMPKα, Phospho-Acetyl-CoA Carboxylase (Ser79) antibodies were from Cell Signaling (Berverly, Mass., USA). Antibodies against Fibronectin were from BD Bioscience (Pont de Claix, France). Antibody to human SPARC was purchased from Hematologic Technologies (Essex Junction, Vt., USA). Antibody to human N-Cadherin was purchased from Invitrogen (South Washington, D.C., USA). Antibody to human S100 was purchased from Abcam (Cambridge, Mass., USA).

Cell Cultures:

Different cancer cell lines were purchased from American Tissue Culture Collection (Molsheim, France). Cells were grown in RPMI 1640 (A375, WM9, SKMel28 and LNCap) or in DMEM medium (1205Lu, Mel501, Mewo and PC3) supplemented with 10% FCS and penicillin/streptomycin (100 U/ml/50 mg/ml) at 37° C. and 5% CO2. Patient melanoma cells were prepared as described (13). Briefly, biopsy was dissected and digested for 1-2 h with collagenase A (0.33 U/ml), dispase (0.85 U/ml) and Dnase I (144 U/ml) with rapid shaking at 37° C. Large debris were removed by filtration through a 70-mm cell strainer. Viable cells were obtained by Ficoll gradient centrifugation.

Small Interfering RNA Transfection:

Transfection of duplex siRNAs (50 nM) was carried out using Lipofectamine RNAiMAX (Invitrogen) in Opti-MEM (Invitrogen). The day after the transfection, metformin was added to the medium and proteins were extracted 24 h after the addition of metformin. Stealth siRNA targeting AMPKα1, AMPKα2, and p53 were purchased from Invitrogen, whereas AMPK siRNA were from Dharmacon (Lafayette, Colo., USA). As nonspecific control, a scramble sequence siRNAs were used.

Infection with Adenovirus:

Adenoviruses encoding a dominant negative form (Ad AMPK-DN) of subunits α1 and α2 of AMPK were a generous gift of Dr. Foufelle (INSERM, UMR-S 872, Paris, France). An adenovirus of which the expression cassette contains the major late promoter with no exogenous gene was used as control (Ad control). Adenoviruses were propagated in human embryonic kidney 293 cells and stored at −80° C. 1205Lu cells were infected for 24 h with the Ad AMPK-DN prior to the metformin treatment.

Luciferase Assays:

Melanoma cells were seeded in 24-well dishes, and transient transfections were performed the following day using 2 μl Lipofectamine (Gibco-BRL, Eragny, France) and 0.3 mg of PG13-Luc, a p53-dependent firefly luciferase reporter gene in a 200-ml final volume. pCMVβGal plasmid was cotransfected to control the variability of transfection efficiency in the reporter assays. The day after the transfection, metformin was added to the medium. At 24 hours after stimulation, cells were harvested in 50 μl of lysis buffer and soluble extracts assayed for luciferase and β-galactosidase activities. All transfections were repeated several times using different plasmid preparations. Luciferase assays were carried out exactly as described (17).

Western Blot Assays:

Protein were extracted in buffer containing TRIS-HCl pH7.5 50 mM, NaCl 15 mM, Triton X-100 1% and protease and phosphatase inhibitor 1X. Briefly, cell lysates (30 μg) were separated by SDS-PAGE, transferred onto a PVDF membrane (Millipore, Molsheim, France) and then exposed to the appropriate antibodies. Proteins were visualized with the ECL system from Amersham (Arlington, Heights, Ill., USA). The western blots shown are representative of at least 3 independent experiments.

Co-Immunoprecipitation Assay:

For the coimmunoprecipitation experiments, 1205Lu melanoma cells were treated 24 h with 10 mM of metformin and lysed in Fischer buffer. 50 μl of protein G agarose (Invitrogen) were mixed with 2 μg of monoclonal anti-p53 antibody for 2 h at 4° C. Then lysates were added and mixed at 4° C. over night. The immunoprecipitated complexes were analyzed by 10% SDS-PAGE and immunoblotting using anti-AMPKα antibody and anti-p53 antibody.

Reverse Transcription and Quantitative PCR:

Total cell RNA was extracted using the RNAeasy miniprep kit (Qiagen), according to the manufacturer's instructions, and 2 μg of RNA was reverse amplified using oligo dT using reverse transcription system (Promega), according to manufacturer's instructions. PCR was performed using StepOnePlus real time PCR system, and the power SYBR green PCR master mix reagent (Applied biosystems, Foster city, CA, USA). Relative quantification of the amplicons was performed by 2(-Delta Delta C(T)) method.

Migration Assay:

Boyden chambers (8.0-μm pores, Transwell, Corning, Inc.) were placed into 24-well chambers containing medium supplemented with 10% FCS. The cells were resuspended in FCS-starved medium, loaded into the top chamber. 4 h later, cells adherent to the underside of the filters were fixed with 4% PFA and stained with 0.4% crystal violet, and five random fields at 20× magnification were counted. Results represent the average of triplicate samples from three independent experiments.

Invasion Assay:

Boyden chambers (8.0-μm pores, Transwell, Corning, Inc.) were coated with 1 mg/ml Matrigel® (BD Biosciences) and were placed into 24-well chambers containing medium supplemented with 10% FCS. The cells were resuspended in FCS-starved medium, loaded into the top chamber. 5 h later, cells adherent to the underside of the filters were fixed with 4% PFA and stained with 0.4% crystal violet, and five random fields at 20× magnification were counted. Results represent the average of triplicate samples from three independent experiments.

Three-Dimensional Spheroid Growth:

Melanoma spheroids were prepared using the liquid overlay method. Briefly, 500 μL of melanoma cells (20000/ml) were added to a 24-well plate coated with 1.5% agar (Difco, Sparks, Md.). Plates were left to incubate for 72 hours, by which time cells had organized into three-dimensional spheroids. Spheroids were then harvested using a P1000 pipette. The medium was removed and the spheroids were implanted into a gel of bovine collagen I containing MEM (Invitrogen). Normal melanoma medium was overlaid on top of the solidified collagen. After different time, pictures of the invading spheroids were taken using a Zeiss microscope.

Matrix Metalloproteinase (MMP) Activity Measurement:

The culture media from stimulated cells were harvested and incubated in a 96-well plate with 0.2 mM of NH2-RA-Dpa-LGLP-AMC as a substrate for various times at 37° C. MMP activity was measured in quadruplicate by quantifying the emission at 460 nm (excitation at 390 nm) in the presence or absence of 10 μM CP471474. The enzyme activities were expressed in arbitrary units per mg of protein.

Substrate Zymography:

The culture media from 1205Lu melanoma cells was concentrated in centrifugal filter unit and loaded on 10% SDS-polyacrylamide gels containing 1 mg/ml type I collagen (BD Biosciences). To estimated the protein concentration, 1205Lu melanoma cells were lysed in a buffer containing 1% Triton X-100, 150 mM NaCl, and 20 mM Tris, pH 7.4, supplemented with a protease inhibitor mixture (Complete EDTA-free, Roche Molecular Biochemicals) at 4° C. under agitation for 30 min. Lysates were clarified by brief spinning, and protein concentration was evaluated by bicinchoninic acid technique (BCA protein assay kit, Pierce). Following electrophoresis, proteins were renatured by incubating gels in 2.5% Triton X-100 for 2 h at 37° C. Gels were then washed three times in distilled water and incubated in substrate buffer (50 mM Tris, pH 7.4, and 1 mM CaCl2) at 37° C. for 24 h with gentle shaking Gels were stained with 0.1% Coomassie Blue R-250 (Sigma) and destained in 7% acetic acid. Enzymatic activities appear as cleared bands in a dark background.

In Vivo Studies:

1205Lu cells stably transfected with a vector encoding luciferase cells come from the team of Dr. Tartare-Deckert. A total of 1×106/150 μl PBS 1205Lu-Luc cells were injected via the tail vein of nude mice (Harlan Laboratories). The mice were treated with or without intraperitoneal injection of 60 mg/kg metformin each day. Melanoma cells were visualized in the animal after intraperitoneal injection of 50 mg/kg luciferin (Caliper Life Sciences) by bioluminescence imaging using a Photon Imager (Biospace Lab). Mice were killed and the lungs were excised, fixed, and serially sectioned. S100 (1/100) and Slug (1/100) immunostaining was performed. To perform pulmonary extravasation analysis, 1.5×106 1205Lu cells were labelled for 1 h with CellTracker™ Green (Invitrogen) and injected via the tail vein of nude mice. After 24 h mice were sacrificed, and the lungs were harvested for analysis with a Zeiss Inverted Scope.

Statistical Analysis:

Results are presented as mean±SE with experiment numbers indicated in the figure legends. Statistical significance was assessed using the Student's t-test. P≦0.05 was accepted as statistically significant.

Results

Metformin Inhibits Cell Invasion but not Cell Migration:

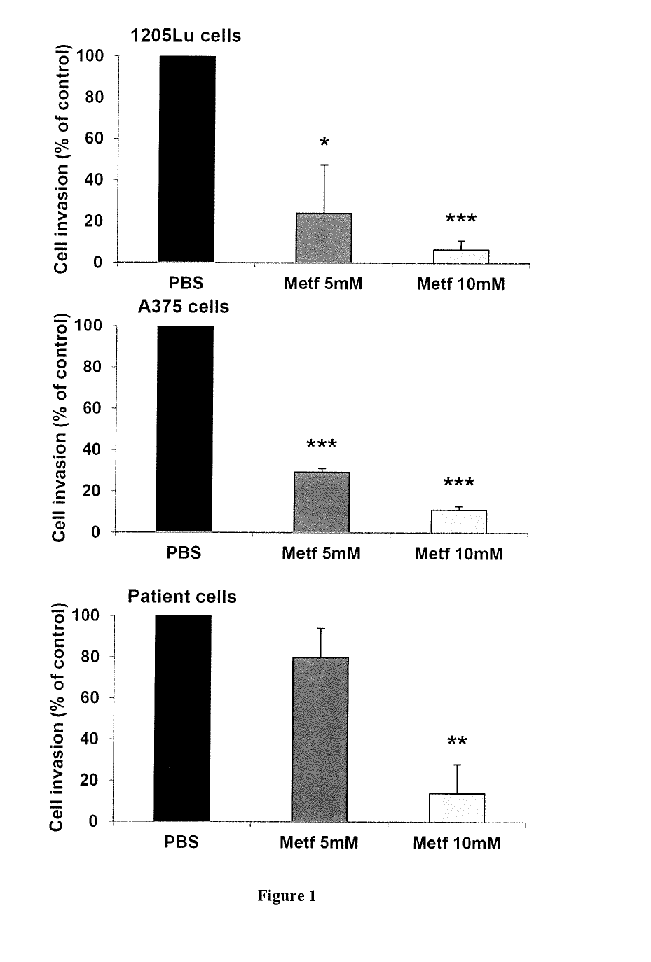

We previously demonstrated that the antidiabetic drug, metformin induced cell death of melanomas cells after long term treatment of 96 hours (13). We now determine whether metformin are able to inhibit migration and invasion properties of melanoma cells at early times. As presented in cell migration assay using Boyden chambers, metformin do not inhibit migration of both melanoma cell lines 1205Lu and A375 after 24 hours. Results were confirmed using wound healing assay. We next determine the capacity of metformin to inhibit cell invasion using Boyden chamber coated with matrigel (FIG. 1). Metformin decreases cell invasion in dose dependent manner in both melanoma cell lines, 1205Lu and A375. At concentration of 10 mM, metformin inhibits by 95% and 90% cell invasion in 1205Lu and A375 cells respectively. Similar results were obtained with cells freshly isolated from patients with significant inhibition of cell invasion in condition with metformin 10 mM.

Tumor invasion was then analyzed in a more physiological context; WM9 melanoma cells were grown as spheroids embedded in collagen. Metformin significantly reduced cell invasion into collagen. To confirm that invasion inhibition is not due to apoptosis induced by metformin, we performed same experiment in presence of apoptosis inhibitor, QVD. As expected, contrary to QVD alone, association of QVD with metformin block invasion indicating that apoptosis does not account for the inhibitory effects on cell invasion mediated by metformin.

Metformin Decreases Expression of Proteins Involved in Epithelial Mesenchimal Transition (EMT):

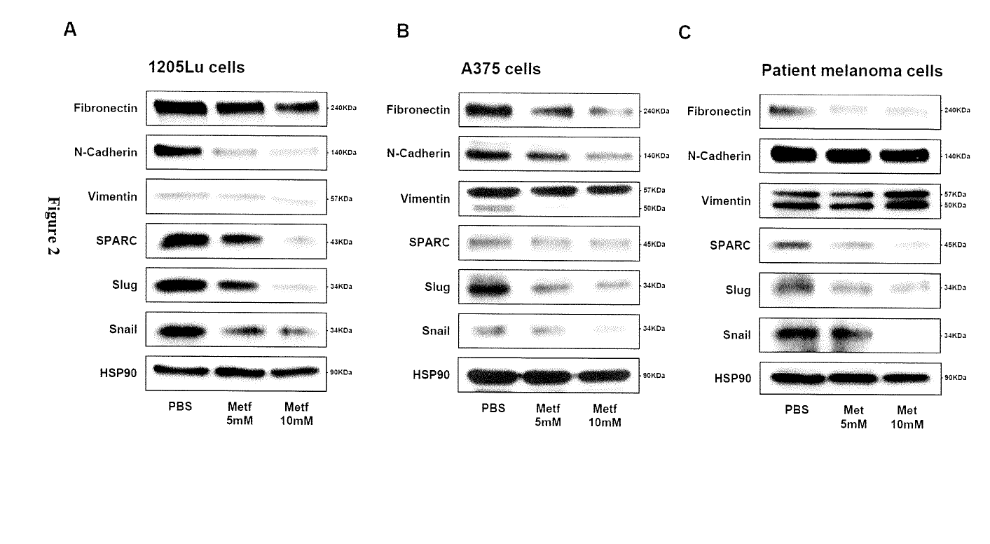

To determine proteins involved in the inhibition of invasion mediated by metformin, we checked by western blot analysis expression of proteins involved in EMT. Metformin inhibited in a dose dependent manner expression of key proteins involved in this process such as fibronectin, N-cadherin or SPARC in 1205 melanoma cells (FIG. 2A). In contrast, vimentin expression was not modified by metformin. Levels of both transcription factors Slug and Snail that initiate EMT was also decrease. Similar results were found in A375 melanoma cells and in isolated patient melanoma cells (FIGS. 2B and 2C respectively).

Metformin Inhibits Activation of Matrix MMPs in Melanoma Cells:

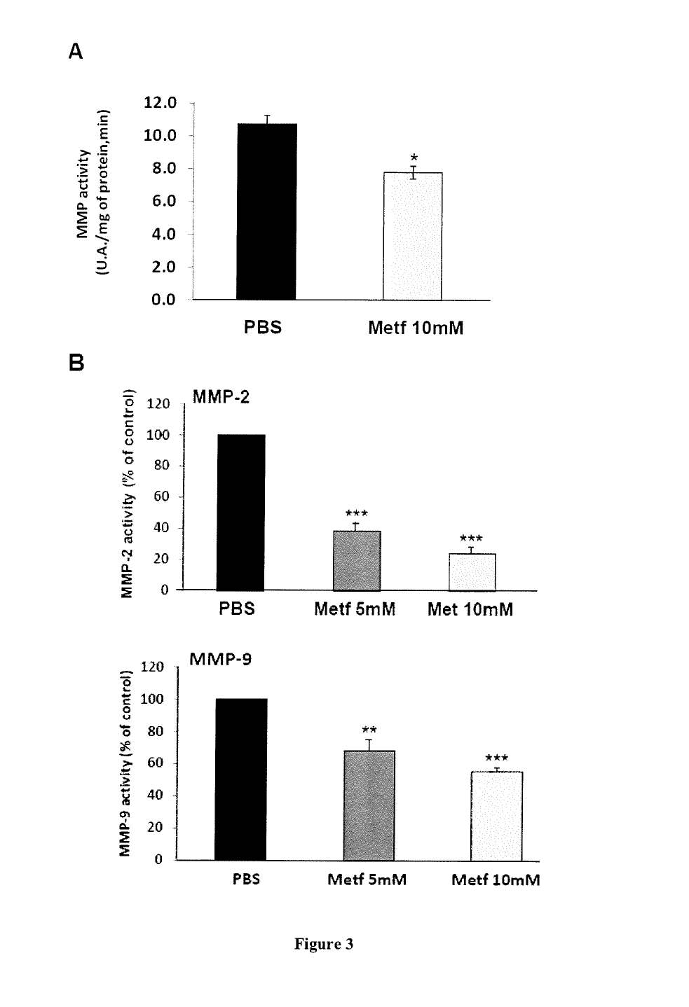

We next examined MMP activities in melanoma cells treated with metformin. Total MMP activity level was assessed using a broad-spectrum fluorogenic MMP substrate on 1205Lu melanoma cells treated by metformin (FIG. 3A). Metformin 10 mM induced a slight but significant decrease of approximately 30% of total MMP activities. In addition, cell-associated metalloproteinase activities were assessed by type I collagen substrate gel zymography. Collagen zymography allowed the detection of enzymatic activities at 82 and 62 KDa that are consistent with active forms of MMP-9 and MMP-2, respectively. In basal condition (PBS), activities were high for both MMPs. Quantification shows an important diminution in response to 5 mM metformin for both MMP to reached 80% and 45% of decrease at 10 mM metformin for MMP-2 and MMP-9 respectively (FIG. 3B).

AMPK is Involved in Inhibition of Invasion Mediated by Metformin:

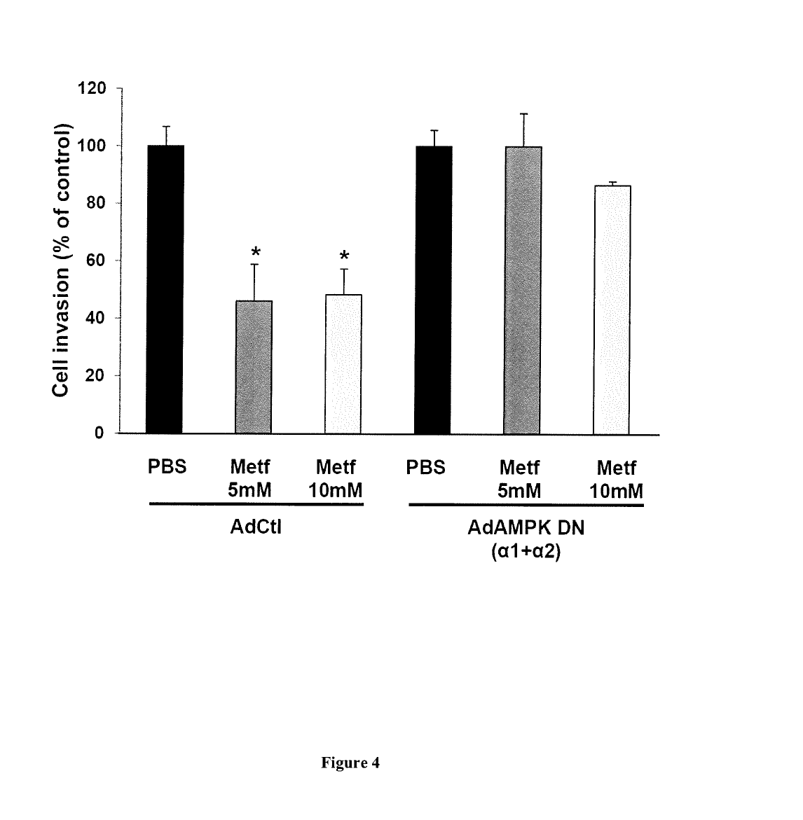

To determine if AMPK activation play a role in inhibition of invasion by metformin, we abrogate AMPK activation by metformin using infection of dominant negative adenoviruses forms of AMPK (AdAMPK DN) in 1205Lu melanoma cells (FIG. 4). As expected, infection of AdAMPK DN α1 and α2 increases the expression of AMPK α1 and α2 indicating that dominant negative forms of AMPK are expressed in the cells. Further, basal and metformin-stimulated phosphorylation of direct AMPK substrate, Acetyl-CoA carboxylase (ACC) is abolished in cells infected by AdAMPK DN α1 and α2 demonstrating that AMPK activation is inhibited.

In parallel, we observed that Slug and SPARC are inhibited in response to metformin in cell infected with Ad control. In contrast, expression of AMPK DN constructs abrogates these inhibitory effects.

Finally, we tested a capacity of metformin to inhibited invasion using Boyden chamber in presence (Ad control) or absence (AdAMPK DN α1 and α2) of active AMPK. Interestingly, metformin-induced inhibition of invasion was abolished in presence of dominant negative forms of AMPK. Taken together these results suggest an implication of AMPK in the inhibitory effect of metformin in invasion.

Transcription Factor p53 is Involved in Inhibition of Invasion Mediated by Metformin:

Like AMPK is involved in p53 activation, we wondered whether this transcription factor could play a role in inhibition of invasion mediated by metformin. Firstly, we verified that in our system, metformin is able to activate p53. For this, reporter assay using a promoter-luciferase construct that contains p53-binding sites, revealed that treatment with metformin 5 and 10 mM led to approximately tenfold and twentyfold induction of p53 promoter activity respectively. As expected, Actinomycin D (ActD) used as positive control of p53 activation leads to an increase of p53 promoter activity comparable to metformin 10 mM.

We next wanted to determine whether upon metformin stimulation of melanoma cells, AMPKα could associate with p53 to induce p53 activation. We immunoprecipitated p53 from 1205Lu cells stimulated or not with metformin (10 mM) for 24 h. Proteins were then blotted with antibodies to either p53 or AMPKα. In unstimulated conditions, p53 is poorly associated with AMPKα, but after metformin treatment, a large increased amount of p53 is co-immunoprecipitated with AMPKα. As control, total blot were presented and show no major modification of AMPKα level and a decrease in Slug, N-cadherin and fibronectin expressions in response to metformin. We conclude that in intact 1205Lu melanoma cells, p53 associates with AMPKα in a metformin-dependent fashion.

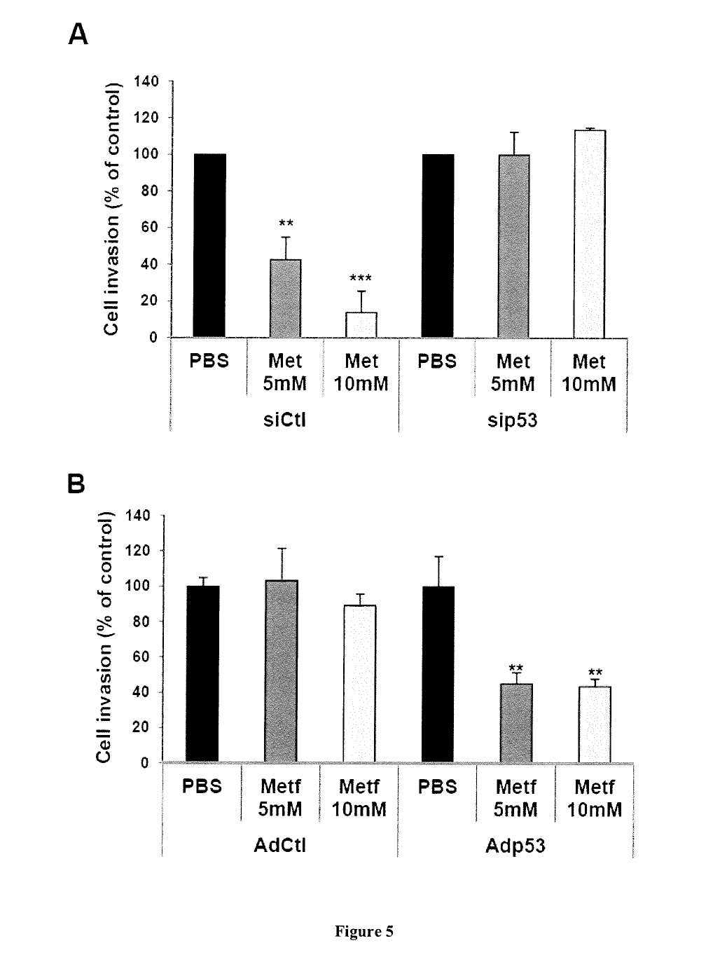

Further we asked whether decreasing level of p53 could prevent inhibition of invasion induced by metformin. We observed that siRNA-mediated downregulation of p53 prevent downregulation of Slug and inhibition of invasion induced by metformin (FIG. 5A). Similar results were obtained in other melanoma cell line, Mel501 stably transfected with shp53 RNA.

Regarding a functional role for p53 in mediating anti-invasion properties of metformin, melanoma cells harboring a mutated TP53 gene (Mewo, SKme128 and HMV2 cells) exhibited resistance to metformin mediated inhibition of invasion using western blot analyses and Boyden chamber assay (FIG. 5B). Interestingly, re-expression of WT p53 expression by adenoviruses infection in Mewo cells re-sensitizes cells to metformin (FIG. 4B) and restored the inhibition of invasion, the decrease in Slug and N-cadherin expressions, in response to metformin.

Our results show that inhibition of invasion induced by metformin occurs through an AMPKα/p53-dependent mechanism.

Metformin Inhibits Melanoma Metastasis Development in Mice Using Extravasation and Metastasis Models:

Finally, to assess a potential anti-metastasis effect of metformin in vivo, extravasation and lung metastasis models were performed in immunodeficient nude mice.

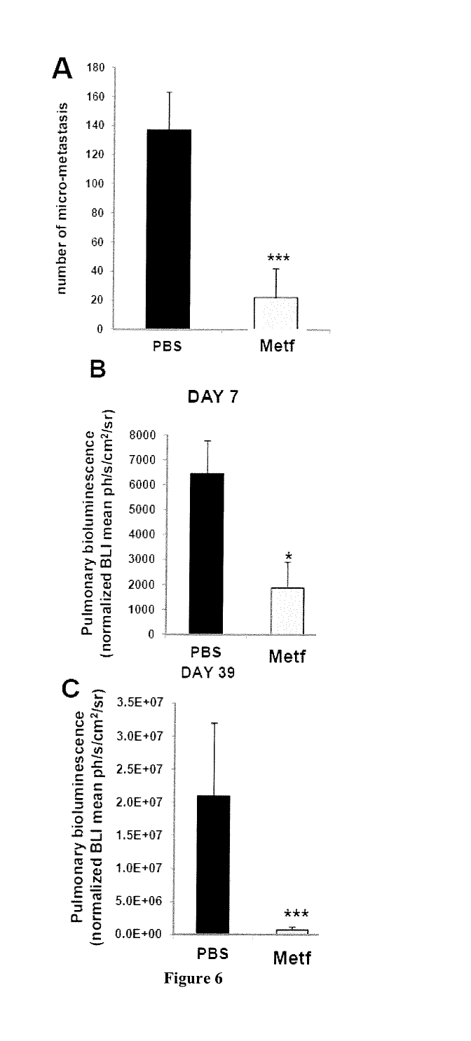

Green-labeled human melanoma cells 1205Lu treated or not 24 h by metformin were injected in the caudal vein of 6-week-old female athymic nude mice, and their ability to extravasate through the pulmonary parenchyma was evaluated (FIG. 6A). As shown in figure, the control 1205Lu cells treated by PBS give much more micro-metastases in the lungs than 1205Lu cells treated by metformin. Quantification of experiments by counting extravasated cells using inverted microscope confirmed this result. In addition, similar experiment performed with Mewo cells harboring p53 mutation shows the insensibility of metformin to inhibit extravasation in lungs confirming the implication of p53 in this process in vivo.

In other experiment, 1205Lu melanoma cells (1.5×10⁶) stably expressing luciferase were injected in caudal vein of 6-week-old female athymic nude mice and were then treated daily with an intraperitoneal injection of vehicle or metformin (2 mg/mouse/day) over a period of 39 days (FIG. 6B). 7 days after cell injection, bioluminescence was detected in the lungs of all mice. Importantly, a 3-fold increase in bioluminescence intensity was observed in the lungs of mice treated with vehicle compared to lungs of mice treated by metformin. This result reflects the decreased capacity of cells of mice treated by metformin to metastase in lung in vivo. After 39 day, bioluminescence intensity was very weak in lungs of mice treated by metformin in comparison of lungs of control mice (FIG. 6C). This inhibition was not found when we used Mewo cells with inactive p53.

To confirm the molecular mechanisms involved in the antimetastasis effects of metformin in vivo, Slug expression was studied by immunofluorescent staining on tumor sections from mice treated with vehicle or metformin (FIG. 6C). S100 staining was used to detect melanoma cells in lungs. Sections of lung tumors from mice treated with metformin show a significant decrease in Slug staining compared with sections of tumors from control mice injected with vehicle. Quantification of ration Slug/S100 confirms this observation. Thus, the reduction of metastases observed in metformin-treated mice seems to be, at least in part, related to the inhibition of the expression of Slug protein.

### Example 2

### Other AMPK Activators Block Melanoma Invasion and Metastasis Development in a p53-Dependent Manner

Material & Methods

Melanoma cell lines were cultured as described in Example 1. Invasion assay was carried out by as described in Example 1.

Results

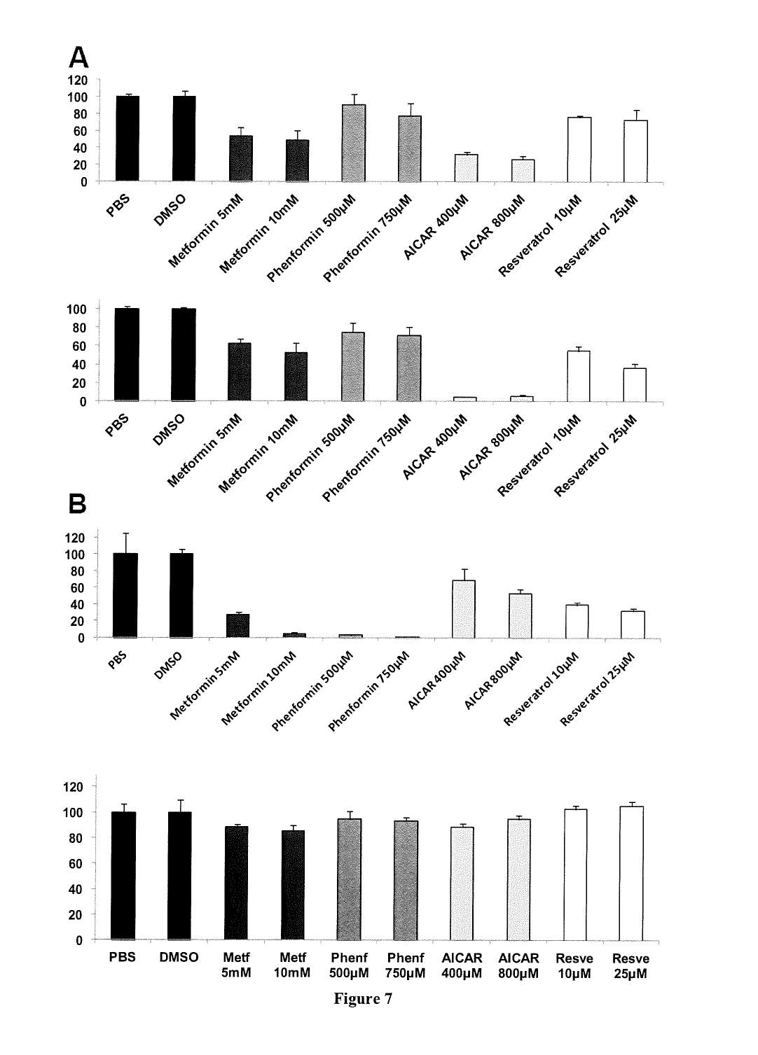

We next determine the capacity of other AMPK activators such as phenformin, AICAR and resveratrol to inhibit cell invasion using Boyden chamber coated with matrigel (FIG. 7A). AMPK activators decreases cell invasion in dose dependent manner in both melanoma cell lines, 1205Lu and A375.

### Example 3

### AMPK Activators Block Prostate Cancer Invasion and Metastasis Development in a p53-Dependent Manner

Material & Methods

Prostate cancer cell lines were cultured as described in Example 1. Invasion assay was carried out by as described in Example 1.

Results

We also determine the capacity of AMPK activators to inhibit cell invasion in p53-mutated and -non mutated prostate cancer cells (FIG. 7B). Contrary to prostate cancer cells mutated on p53, PC3, AMPK activators decrease cell invasion in prostate cancer cells with WT p53 (LNCap). These results indicate that, like in melanoma cells, p53 is necessary for inhibition of invasion mediated by AMPK activators.

### Example 4

### AMPK Activators Block Lung Cancer Invasion and Metastasis Development

Material & Methods

Lung cancer cell line A459 was cultured as described in Example 1. Invasion assay was carried out by as described in Example 1.

Results

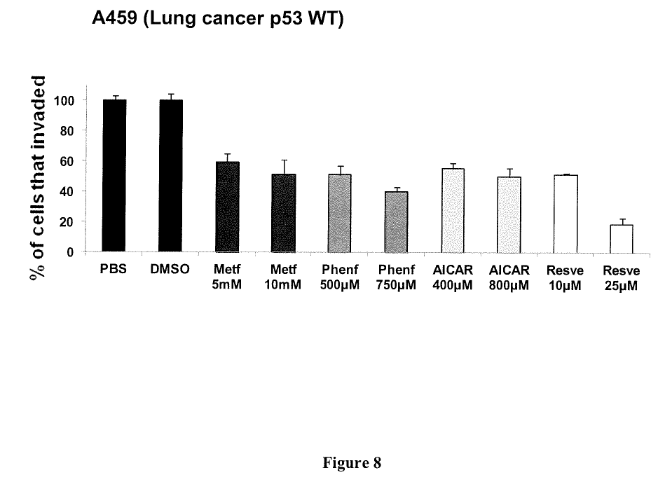

We finally determine the capacity of AMPK activators to inhibit cell invasion in p53-non mutated prostate cancer cells (FIG. 8). AMPK activators decrease cell invasion in lung cancer cells with WT p53 (A459).

### REFERENCES

Throughout this application, various references describe the state of the art to which this invention pertains. The disclosures of these references are hereby incorporated by reference into the present disclosure.
1. Demierre M F. Epidemiology and prevention of cutaneous melanoma. Curr Treat Options Oncol. 2006; 7:181-6.
2. Fukunaga-Kalabis M, Santiago-Walker A, Herlyn M. Matricellular proteins produced by melanocytes and melanomas: in search for functions. Cancer Microenviron. 2008; 1:93-102.
3. Hsu M Y, Wheelock M J, Johnson K R, Herlyn M. Shifts in cadherin profiles between human normal melanocytes and melanomas. J Investig Dermatol Symp Proc. 1996; 1:188-94.
4. Nieto M A. The snail superfamily of zinc-finger transcription factors. Nat Rev Mol Cell Biol. 2002; 3:155-66.
5. Sefton M, Sanchez S, Nieto M A. Conserved and divergent roles for members of the Snail family of transcription factors in the chick and mouse embryo. Development. 1998; 125:3111-21.
6. Gupta P B, Kuperwasser C, Brunet J P, Ramaswamy S, Kuo W L, Gray J W, et al. The melanocyte differentiation program predisposes to metastasis after neoplastic transformation. Nat Genet. 2005; 37:1047-54.
7. Wu W S, Heinrichs S, Xu D, Garrison S P, Zambetti G P, Adams J M, et al. Slug antagonizes p53-mediated apoptosis of hematopoietic progenitors by repressing puma. Cell. 2005; 123:641-53.
8. Wang S P, Wang W L, Chang Y L, Wu C T, Chao Y C, Kao S H, et al. p53 controls cancer cell invasion by inducing the MDM2-mediated degradation of Slug. Nat Cell Biol. 2009; 11:694-704.
9. Miller A J, Mihm M C, Jr. Melanoma. N Engl J Med. 2006; 355:51-65.
10. Ben Sahra I, Le Marchand-Brustel Y, Tanti J F, Bost F. Metformin in cancer therapy: a new perspective for an old antidiabetic drug? Mol Cancer Ther. 2010; 9:1092-9.
11. Pollak M. Metformin and other biguanides in oncology: advancing the research agenda. Cancer Prev Res (Phila). 2010; 3:1060-5.
12. Decensi A, Puntoni M, Goodwin P, Cazzaniga M, Gennari A, Bonanni B, et al. Metformin and cancer risk in diabetic patients: a systematic review and meta-analysis. Cancer Prev Res (Phila). 2010; 3:1451-61.
13. Tomic T, Botton T, Cerezo M, Robert G, Luciano F, Puissant A, et al. Metformin inhibits melanoma development through autophagy and apoptosis mechanisms. Cell Death Dis. 2011; 2:e199.
14. Janjetovic K, Harhaji-Trajkovic L, Misirkic-Marjanovic M, Vucicevic L, Stevanovic D, Zogovic N, et al. In vitro and in vivo anti-melanoma action of metformin. Eur J Pharmacol. 2011; 668:373-82.
15. Niehr F, von Euw E, Attar N, Guo D, Matsunaga D, Sazegar H, et al. Combination therapy with vemurafenib (PLX4032/RG7204) and metformin in melanoma cell lines with distinct driver mutations. J Transl Med. 2011; 9:76.
16. Woodard J, Platanias L C. AMP-activated kinase (AMPK)-generated signals in malignant melanoma cell growth and survival. Biochem Biophys Res Commun. 2010; 398:135-9.
17. Rocchi S, Picard F, Vamecq J, Gelman L, Potier N, Zeyer D, et al. A unique PPARgamma ligand with potent insulin-sensitizing yet weak adipogenic activity. Mol Cell. 2001; 8:737-47.

1. An in vitro method for predicting the responsiveness of a patient suffering from a cancer to a prophylactic treatment with an 5′ adenosine monophosphate-activated protein kinase (AMPK) activator suitable for use in preventing metastasis, said method comprising a step of determining the presence of a mutated p53 gene or a mutant form of the p53 protein in a tumor biopsy obtained from said patient, wherein if a mutated p53 gene or a mutant form of the p53 protein is present in said biological sample, then non-response of the patient to the prophylactic treatment with an AMPK activator is indicated, but if a mutated p53 gene or a mutant form of the p53 protein is not present in said biological sample, then a response of the patient to the prophylactic treatment with an AMPK activator is indicated.

2. (canceled)

3. The method according to claim 1, wherein the p53 gene mutation leading to said mutated p53 gene or said mutant form of the p53 protein is selected from the group consisting of missense mutations, nonsense mutations and frameshift mutations.

4. The method according to claim 3, wherein the missense mutation is a missense mutation affecting residues within the p53 DNA-binding domain.

5. The method according to claim 1, wherein the p53 gene mutation leading to said mutated p53 gene or said mutant form of the p53 protein is a loss-of-function mutation.

6. The method according to claim 1, wherein said mutation is detected by using an amplification assay, a hybridation assay, by molecular cloning and sequencing, by microarray analysis or by any method used for determining the presence of a mutation within a DNA sequence or of a mutated form of a protein.

7. An in vitro method for predicting the responsiveness of a patient suffering from a cancer to a prophylactic treatment with an AMPK activator suitable for use in preventing metastasis, said method comprising a step of determining the presence of a wild-type p53 gene or wild-type p53 protein in a tumor biopsy obtained from said patient wherein the presence of a wild-type p53 gene or wild-type p53 protein in said tumor biopsy is indicative of the response of the patient to the prophylactic treatment with an AMPK activator.

8. The method according to claim 1, wherein said cancer is melanoma.

9. A method of preventing metastasis in a patient suffering from a cancer, wherein said patient has a non-mutated p53 gene or lacks a mutant form of the p53 protein in a tumor biopsy obtained from said patient, comprising
administering to said patient a therapeutically effective amount of an AMPK activator, wherein said therapeutically effective amount prevents said metastasis in said patient.

10. A method of improving the survival time of a patient suffering from a cancer, wherein said patient has a non-mutated p53 gene or lacks a mutant form of the p53 protein in a tumor biopsy obtained from said patient, comprising
administering to said patient a therapeutically effective amount of an AMPK activator, wherein said therapeutically effective amount improves said survival time of said patient.

11. The method of claim 9, wherein said AMPK activator is selected from the group consisting of biguanide derivatives, stilbene derivatives, thiazolidinedione (TZD) derivatives, 5-aminoimidazole-4-carboxamide-1-β-D-ribofuranoside (AICAR), thienopyridone derivatives, imidazole derivatives and thiazole derivatives.

12. The method according to claim 11, wherein said biguanide derivative is metformin.

13. The method of claim 9, wherein said patient suffering from cancer is treated with a chemotherapeutic agent against said cancer.

14. The method of claim 9, wherein said cancer is melanoma.

15. The method of claim 10, wherein the survival time is Progression-Free Survival (PFS).

16. The method of claim 10, wherein the survival time is Overall Survival (OS).

17. A kit-of-part comprising an AMPK activator and a p53 recombinant protein or a polynucleotide encoding said p53 recombinant protein.

18. The kit-of-part according to claim 17, wherein the AMPK activator is a biguanide derivative.

19. The kit-of-part according to claim 18, wherein the biguanide derivative is metformin.

20. The kit-of-part according to claim 17, further comprising means suitable for determining the presence of a wild-type p53 gene or wild-type p53 protein and/or the presence of a mutated p53 gene or a mutant form of the p53 protein in a tumor biopsy obtained from a patient.

21. The kit-of-part according to claim 20, wherein said means are primers suitable for amplifying nucleic acid corresponding to the p53 gene.

22. A kit-of-part comprising an AMPK activator and a p53 recombinant protein or a polynucleotide encoding said p53 recombinant protein for simultaneous, separate or sequential use in preventing metastasis in a patient suffering from a cancer, wherein said patient has mutated p53 gene or a mutant form of the p53 protein.

23. The kit-of-part for use according to claim 22, wherein the AMPK activator is a biguanide derivative.

24. The kit-of-part for use according to claim 23, wherein the biguanide derivative is metformin.

25. The method of claim 10, wherein said AMPK activator is selected from the group consisting of biguanide derivatives, stilbene derivatives, thiazolidinedione (TZD) derivatives, 5-aminoimidazole-4-carboxamide-1-β-D-ribofuranoside (AICAR), thienopyridone derivatives, imidazole derivatives and thiazole derivatives.

26. The method of claim 10, wherein said patient suffering from cancer is treated with a chemotherapeutic agent against said cancer.

27. The method of claim 10, wherein said cancer is melanoma.
